(19) United States
(12) Patent Application Publication
(10) Pub. No.: US 2008/0077038 A1
(43) Pub. Date: Mar. 27, 2008

(54) Respiratory Volume/Flow Gating, Monitoring, and Spirometry System for Mri

(75) Inventors: Joseph M. McDonough, Philadelphia, PA (US); Raanan Arens, White Plains, NY (US); Sanghun Sin, Bronx, NY (US)

Correspondence Address: RATNERPRESTIA, P O BOX 980, VALLEY FORGE, PA, 19482-0980, US

(73) Assignee: CHILDREN'S HOSPITAL OF PHILADELPHIA, Philadelphia, PA (US)

(21) Appl. No.: 11/666,976

(22) PCT Filed: Oct. 31, 2005
(86) PCT No.: PCT/US05/39539

§ 371 Date: Nov. 13, 2007

Related U.S. Application Data
(60) Provisional application No. 60/624,294, filed on Nov. 2, 2004.

(51) Int. Cl. A61B 5/08

(52) U.S. Cl. 600/538

(57) ABSTRACT

A respiratory volume/flow monitoring system, including a pneumotach that measures micromovements of a membrane in a MRI environment, gates (triggers) a MRI machine to start image acquisition at specific lung volumes or airflow rates during the breathing cycle or other breathing maneuvers of a patient. The system provides breathing motion artifact suppression for any imaging test susceptible to breathing motion artifact, allows respiratory monitoring of the subject during an MRI procedure, and provides the capability to perform spirometric testing while the subject is in the MRI environment. Embodiments of the invention are also applicable for patients undergoing CT imaging or any other imaging modality that allows external triggering.

### FIELD OF INVENTION

The inventive subject matter relates to medical imaging, diagnostics, therapy and intervention. More particularly, this invention relates to devices and methods for monitoring, imaging and diagnosing a patient's breathing in a magnetic resonance imaging (MRI) environment.

### BACKGROUND OF THE INVENTION

Conventional respiratory gating technology for MRI testing consists of tracking the respiratory wave form via a single sensor on a belt encircling the torso of the patient. This sensor is typically a rubber bellows attached on either end to an inelastic belt. As the chest and/or abdomen expands and contracts during breathing, air pressure in the bellows falls and rises as it stretches and contracts. Conventional methods for air flow measurement are not compatible with the magnetic field environment of MR systems. These devices generally involve metallic components, wires, and the close proximity of transducers and computers.

Applicants developed this invention in order to circumvent technical limitations and/or lack of functionality of conventional commercial MRI gating systems. The Applicants discovered that the pressure signal resulting from conventional respiratory gating signals corresponds approximately to the respiratory volume in breath timing, but is subject to many limitations. In particular, the respiratory signal: a) does not provide flow rate; b) is not calibrated while representing volume; c) can exhibit significant time lags with the actual volume changes in the patient; d) is not sensitive to airway obstructive elements that may register as breaths due to torso expansion and not actually involving airflow; and e) may be distorted in patients with thoracic or abdominal deformity.

The Applicants have also discovered that the current systems do not accurately track volume changes in the lungs because the waveform shape is dependent on the placement of the belt, thoracically or abdominally, or in between. Moreover, the waveform shape is dependent on patient position, meaning that if the body position changes, waveform shape may change. Further, the sensitivity of the bellows system signal is affected by the tightness with which the belt is initially placed around the patient.

Other methods of respiratory plethysmography (volume measurement/tracking) that measure chest motion (e.g., inductive, impedance, piezo electric) suffer from the same deficiencies, and require metallic cables and components on the patient. Therefore, there is a need for an optical methodology for the pressure transduction of the flow signal that is compatible for use in the MR environment.

### SUMMARY OF THE INVENTION

The preferred embodiments of the invention eliminate the aforementioned deficiencies of the bellows method and provide additional utility. These benefits are provided because the preferred embodiments measure actual calibrated respiratory volume and flow (preferably through a mask covering the nose and mouth) with high precision and accuracy using microphone technology. In addition, the software allows for gating image acquisition, respiratory motion suppression, monitoring, pulmonary diagnostic spirometry, and data storage of respiratory data for real time and subsequent analysis.

The devices and methods of the preferred embodiment improve upon existing respiratory gating technology for MRI. For example, the improvements allowed MRI images to be gated to specific calibrated lung volumes and/or flow rates for both clinical and research studies. The invention allows triggering of MRI image acquisition during flow limited and obstructive events in the airway. In addition, the preferred embodiments provide accurate and precise respiratory triggering to limit respiratory motion artifact during MRI imaging. The invention also allows for MRI images to be made during specific breathing maneuvers and pulmonary function test. Moreover, the preferred embodiments provide detailed respiratory monitoring information during an MRI procedure and will allow spirometry to be performed during the MRI procedure.

According to a preferred embodiment, a respiratory volume/flow monitoring system, including a pneumotach having an air pressure sensor that measures micromovements of a membrane in a MRI environment, gates (triggers) a MRI machine to start image acquisition at specific lung volumes or airflow rates during the breathing cycle or other breathing maneuvers of a patient. The system provides breathing motion artifact suppression for any imaging test susceptible to breathing motion artifact, allows respiratory monitoring of the subject during an MRI procedure, and provides the capability to perform spirometric testing while the subject is in the MRI environment. The preferred embodiments of this invention are also applicable for patients undergoing CT imaging or any other imaging modality that allows external triggering.

Further scope of applicability of the present invention will become apparent from the detailed description given hereinafter. However, it should be understood that the detailed description and specific examples, while indicating preferred embodiments of the invention, are given by way of illustration only, and that the invention is not limited to the precise arrangements and instrumentalities shown, since the invention will become apparent to those skilled in the art from this detailed description.

### BRIEF DESCRIPTION OF THE DRAWINGS

The following detailed description of preferred embodiments of the invention will be better understood when read in conjunction with the following drawings, in which like referenced numerals designate like elements, and wherein:

FIG. 1 illustrates an optical sensor system in accordance with the preferred embodiment of the invention;

FIG. 2 illustrates a preferred embodiment of the sensor shown in FIG. 1 integrated with a breathing mask;

FIG. 3 illustrates a sectional view of the sensor shown in FIG. 1 in accordance with the preferred embodiment;

FIG. 4 illustrates a sectional view of a sensor in accordance with another preferred embodiment;

FIG. 5 illustrates a schematic of a preferred embodiment of the respiratory gating and monitoring system with a MRI device;

FIG. 6 illustrates exemplary results of the volume triggering provided by a computer program in accordance with the preferred embodiments;

FIG. 7 illustrates a mask and pneumotach system in accordance with yet another embodiment of the invention;

FIG. 8 illustrates an enlarged view of the mask and pneumotach system shown in FIG. 7; and

FIG. 9 illustrates an enlarged view of a breathing mask and pressure sensor in accordance with still another embodiment of the invention.

### DETAILED DESCRIPTION OF THE INVENTION

In accordance with an example of the preferred embodiments, a patient in an MRI scanner wears a mask preferably covering the nose and mouth, and held on the face with straps. The mask is preferably a tightly fitted low profile plastic mask or other modified mask in communication with a pneumotach assembly, including a pressure sense chamber and an air pressure sensor. A port on the mask allows the patient to inhale and exhale room air or other gas mixtures as required. While not being limited to a particular theory, the port is fitted with the pressure sense chamber. The pressure sense chamber includes a laminarizing element, a resistive element and a conduit communicating with the air pressure sensor. The proximal end of the pressure sense chamber includes a laminarizing element having a baffle. The baffle serves to limit turbulence and flow eddies in the pressure sense chamber. The distal end of the pressure sense chamber includes a non-metallic, hydrophobic, porous screen that has a slight linear airflow resistance.

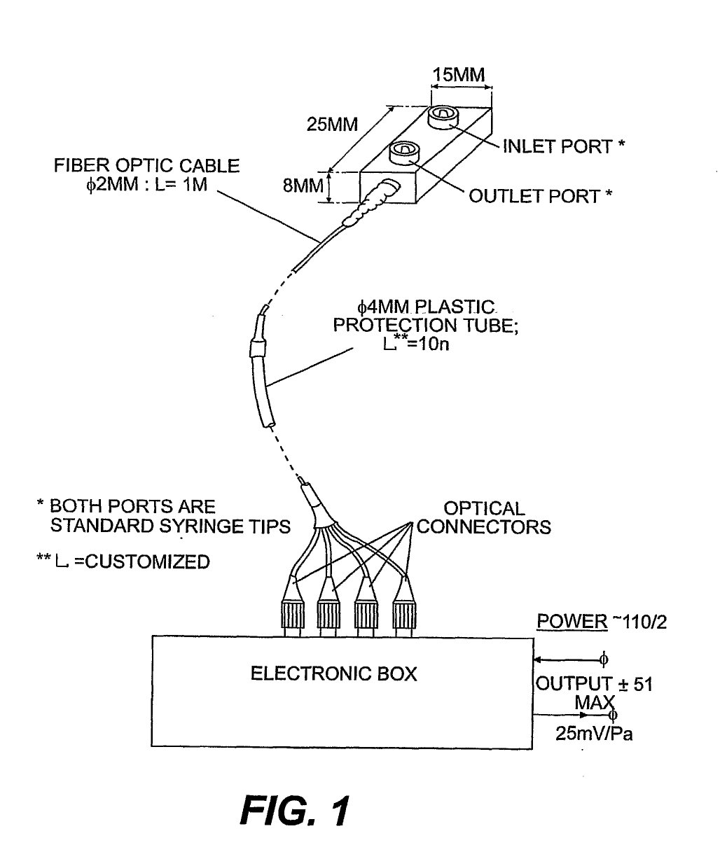

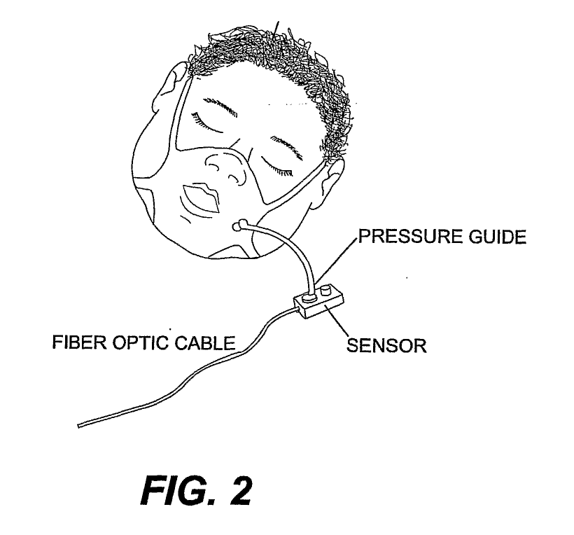

As can best be seen in FIGS. 1 and 2, a pressure guide communicates with the port on the mask (via the pressure sense chamber) and one of the ports of the sensor. With the pressure guide, the inlet and outlet ports of the air pressure sensor do not require the sensor to be at the mask. Short probes (e.g., guides, tubes) can be connected through standard syringe tips. It is also understood that in lieu of covering the nose and mouth, a modified mask could include an endotracheal tube that communicates with the patient's airway.

The sensor is a low pressure optical sensor that benefits from microphone technology. The sensor can work at very low frequencies and can operate in DC. As can best be seen in FIG. 1, the sensor head is preferably a lightweight structure including an inlet port and an outlet port used for measuring pressure difference between the two. In a preferred embodiment, each inlet can be connected to standard syringe tips and therefore can be mounted to measure pressure difference inside and outside the patient's airways, a mask, or at any two points. The fiber optic cables can be customized in terms of length, thickness and type as desired, no signal loss is expected with a length up to 1000 meters. The electro-optic unit includes all optical components and electrical circuitry which provides signal amplifications, conditioning and control loops for stabilization of the sensor output. The output signal is preferably an analog line with a BNC connector. In operation, the electro-optic unit is preferably connected to a main power supply (110 volts).

Still referring to FIG. 1, the pneumotach includes an air pressure sensor, optical fibers, and an electro-optic box that is preferably located in the MRI control room. Preferred specifications for the sensor are as follows:
Pressure range: ±about 200 Pa. The membrane should sense applied pressure relative to ambient on the other side (max pressure: ± about 200 Pa/5.5V).
Self noise: <about 0.2 Pa.
Accuracy: <±about 1 Pa.
Frequency response: about 0-30 Hz.
Operating temperature range: about 17-37 degrees centigrade (Celsius).
Voltage output: ±about 5 volts, corresponding to the ± about 200 Pa pressure range.
Maximum pressure without damaging of membrane: ±about 2000 Pa.

While not being limited to a particular theory, the preferred sensor includes a sensor head, membrane, fibers, electro-optic unit and stabilization electronic circuit. The sensor preferably communicates via a pressure guide with a pressure sense chamber and mask to the patient's airway. The sensor used with the preferred embodiments incorporates optical microphone technology modified to optimally sense air pressure in the range needed by pneumotachography, and is partially derived from the microphone technology as also described in U.S. Pat. No. 5,771,091 to Paritsky et al. and U.S. Pat. No. 5,969,838 to Paritsky et al., the disclosure of which is incorporated by reference herein in its entirety.

The use of the modified optical microphone technology to transduce pneumotach pressure is believed to be new in the arts of pneumotachography and MRI gating. This modified use provides air pressure measurement sensitivities necessary for the accurate gating of a MRI machine to start image acquisition at specific lung volumes or airflow rates during the breathing cycle or other breathing maneuvers. A patient, likely located in a MRI scanner, wears a tightly fitted low profile plastic mask covering the nose and mouth. A pressure guide communicates air from a pressure sense chamber, coupled to the mask, to an inlet port of the pressure sensor. The preferred pressure sensor is a light modulating acoustical sensor that measures micromovements of a membrane caused by changes of air pressure within the sensor.

The pneumotach of the preferred embodiments includes a reflecting sensor and a transducer. The reflecting sensor includes a source of light, such as a light emitting diode (LED) and a photodetector, such as a photodiode or a phototransistor. The transducer transforms the measured physical phenomenon (e.g., air pressure) into a mechanical displacement. Light produced by the LED is guided through a first fiber and illuminates a membrane that includes a reflecting spot. The light is reflected back through a second fiber and the photo detector to a measuring collecting unit, such as, for example, the electronic box shown in FIG. 1. The geometry of the fibers, the optical head, and the membrane are set at angular positions in order to maximize the efficiency of the transformation of membrane movement to light modulation.

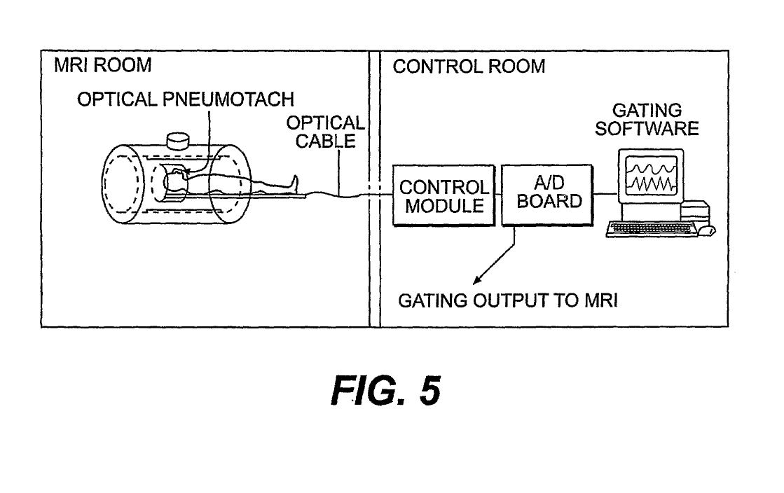

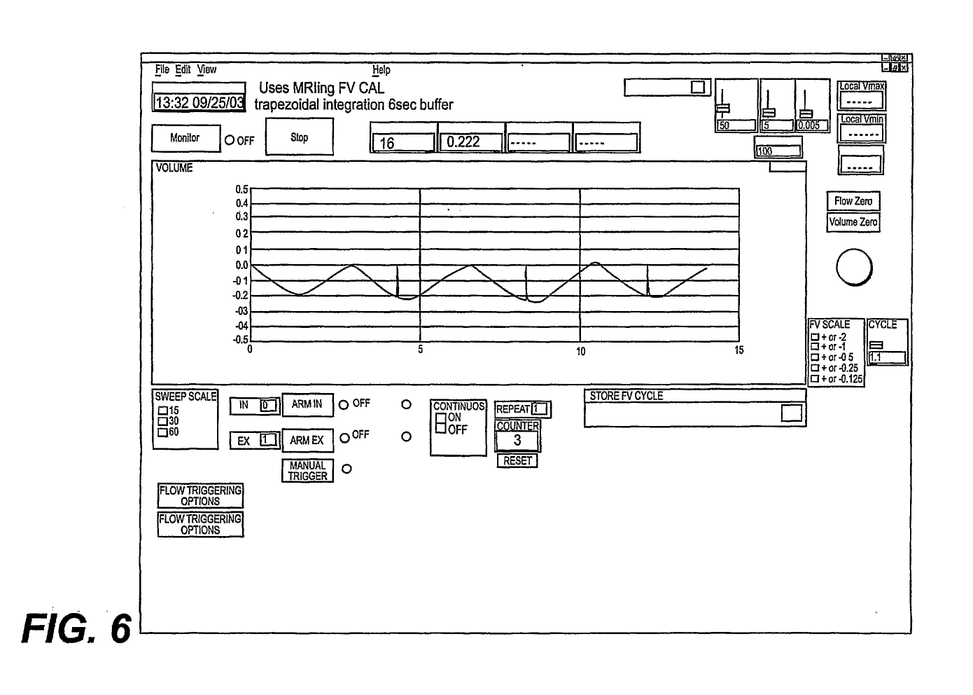

As can best be seen in FIGS. 4 and 5, the pneumotach can include one membrane for the sensor or a plurality of membranes, preferably one membrane for each pressure zone in which the air pressure is measured by the sensor. While not being limited to a particular theory, syringe tips may be coupled to the sensor housing for communicating pressure to the respective pressure zone of the sensor. Since both ports of the system are preferably standard syringe tips, the pressure guide can be connected to the sensor via one of the syringe tips. The other syringe tip may be coupled to ambient pressure or to a different pressure source as desired.

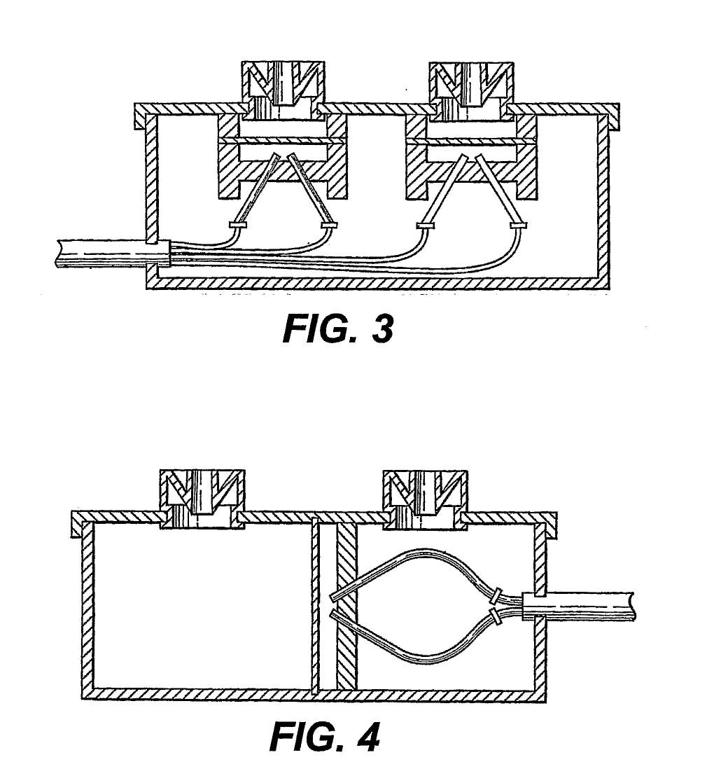

Referring to FIGS. 1-5, during breathing, air pressure in the mask falls on inspiration and rises on expiration, since it passes across the resistive element of the pressure sense chamber. This pressure, which is proportional to air flow, is measured and transduced with the optical pneumotach using optical microphone technology. This transducer methodology eliminates the need for any metallic or conducting cables or transducers to be in the field of the MR magnet. While not being limited to a particular theory, a fiber optic cable communicates the sensor with the electronic box which produces an analog voltage signal proportional to pressure. This analog voltage signal proceeds from the electronic box, which may or may not be in the MRI suite, out of the MRI suite where it is sampled by an analog to digital converter in a computer.

A computer program samples the voltage, converts it to a flow measurement (by a pre-calibrated factor), converts the flow measurement to volume, and displays and stores the waveforms and calculates respiratory parameters in real time. The computer program also sets gating triggers to various volume or flow levels as desired by a user. The gating trigger signal to the MRI machine is made via a digital output channel from the AD board, through a cable, to the gating input port of the MRI machine. The computer program also provides algorithms to zero the sensor to no airflow, adjust for volume drift, reset volume baseline, set pre and post trigger delays for compensation to MR sequences, set monitoring algorithms, and calculate spirometric parameters.

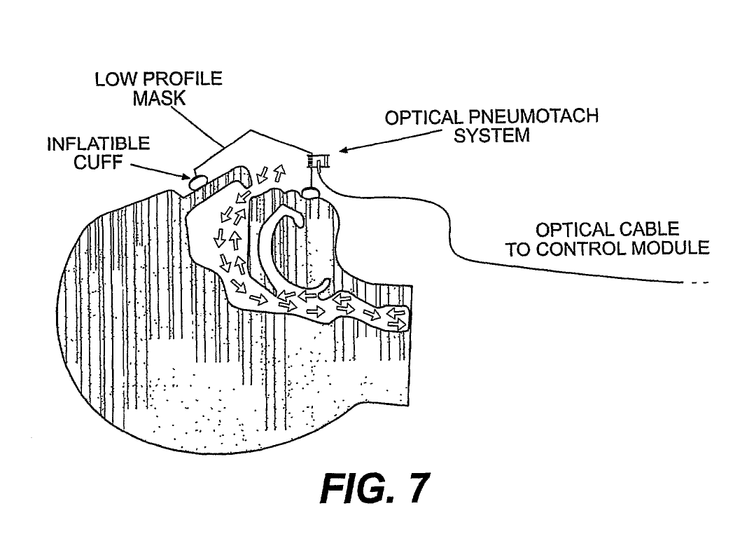

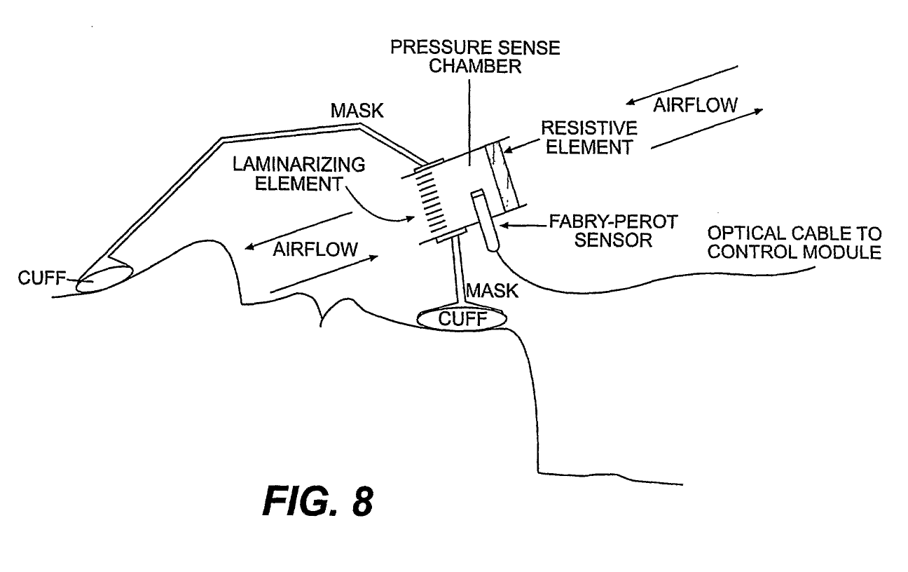

Referring to FIGS. 7 and 8, a non-preferred embodiment is described. While not being limited to a particular theory, a patient in the MRI scanner wears a tightly fitted low-profile plastic mask covering the nose and mouth and held on the face with straps. A port on the mask allows the patient to inhale and exhale room air or other gas mixtures as required. The port is fitted with a cylindrical pneumotach sensor assembly. The proximal end of the pneumotach includes a laminarizing element having a baffle. The baffle serves to limit turbulence and flow eddies in the pressure sense chamber. The distal end of the pneumotach includes a non-metallic, hydrophobic, porous screen that has a slight linear airflow resistance.

During breathing, air pressure in the mask is transduced with an optical pressure sensor fitted into the pressure sense chamber. The sensor in this embodiment preferably utilizes Fabry-Perot Interferometry technology, which eliminates the need for any metallic or conducting cables or transducers to be in the field of the MR magnet. The optical cable proceeds from the chamber out of the MRI room to its control module which produces an analog voltage proportional to pressure. The analog voltage is sampled by an Analog to Digital converter in a PC or laptop computer.

In a similar manner as described above for the preferred embodiments, a software program samples the voltage, converts to it flow (by a precalibrated factor), converts flow to volume, and displays and stores the waveforms and calculates respiratory parameters in real time. Software algorithms allow the user to set gating triggers to various volume or flow levels. The gating trigger signal to the MR machine is made via a digital output channel from the AD board, through a cable, to the gating input port of the MRI machine. The software also provides algorithms to calibrate and zero the pneumotach flow, adjust for volume drift, reset volume baseline, set pre and post trigger delays for compensation to MR sequences, set monitoring alarms, and calculate spirometric parameters.

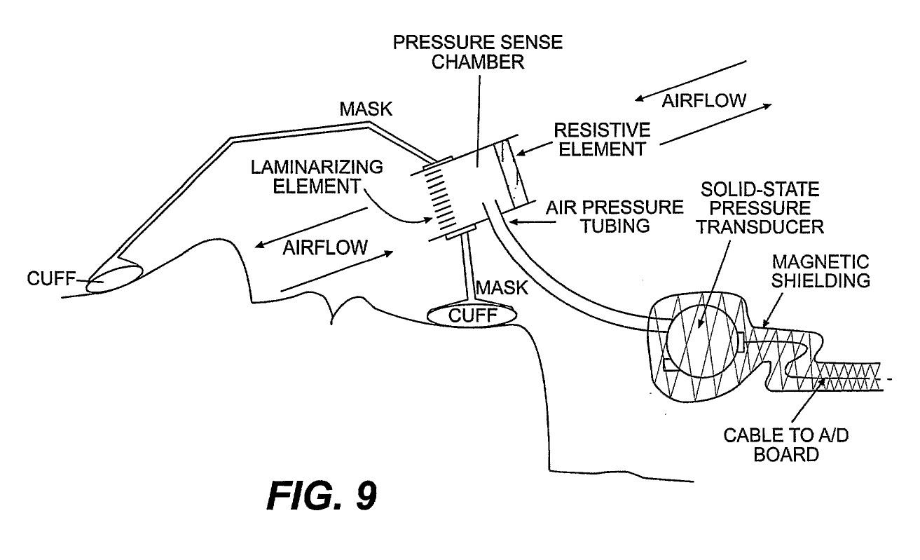

The Fabry-Perot technology may not provide sufficient pressure sensitivity at its current technological maturity. In that case, we propose an alternate method of pressure transduction as shown, for example in FIG. 9. In particular, air-coupled pressure is transduced by a conventional solid-state air pressure transducer placed as closely as possible to, or within the field the MR magnet, but shielded from receiving or creating magnetic interference. The mask pressure is conducted to the pressure transducer via a tube that is preferably plastic, but is not limited thereto. Pressure waveform delays and damping due to the tube conduction can be compensated for in the software algorithms. The invention will allow specific diagnostic imaging modalities to be developed that require accurate and precise respiratory gating. This is beneficial to Radiologic, Cardiac, Pulmonary, and Physiologic diagnostic and research methods.

An exemplary application of the embodiments includes the acquisition of sequential slices at the same point in the breathing cycle in order to produce three dimensional segmentations of the airway showing changes in shape during breathing. Another example includes the imaging of specific point(s) of airway obstruction during obstructive or apneic events as related to respiratory sleep disorders. This can provide diagnostic information as to the cause of the apnea and guide surgical intervention. Yet another example includes testing the effect of a bronchodilator medication by imaging lung airways at times of maximum flow, and concurrently measuring lung function by spirometry. This could provide exact quantification of the drug effect on the airway diameter. Still anther example includes closely monitoring patients susceptible to flow limitation or apnea while having an MRI procedure. Current technology may not provide a sensitive or timely indication of these events, which can lead to significant periods of hypoxia for the subject, before intervention is made.

It will be apparent by those skilled in the art that changes could be made to the embodiments described above without departing from the broad inventive concept thereof. It is understood, therefore, that the invention is not limited to the particular embodiments disclosed, but it is intended to cover modifications within the spirit and scope of the present invention. Without further elaboration, the foregoing will so fully illustrate the invention that others may, by applying current or future knowledge, readily adapt the same for use under various conditions of service.

1. A respiratory volume/flow monitoring system, comprising:
a pneumotach including a housing, at least one pair of optical fibers including a first fiber and a second fiber, each having an input end portion and an output end portion, the input end portion of said first fiber being connectable to a source of light and the output end portion of said second fiber being connectable to a light intensity measuring unit;
a sensor head, including said input end portion of said second optical fiber and said output end portion of said first optical fiber affixedly located at least in proximity to each other;
a membrane that reflects light emerging from the output end portion of said first fiber to said input end portion of said second optical fiber, said second optical fiber measuring the reflected light as a light measurement and forwarding the light measurement via a fiber optic cable to an electric box; and
an airway coupler communicating air having an air pressure to said pneumotach.

2. The system of claim 1, further comprising a control system configured sample an analog voltage proportional to the light measurement, convert the analog voltage to a volume, display and store waveforms associated with the volume and calculate respirator parameters in real time.

3. The system of claim 2, wherein said control system includes an analog to digital (A/D) board and said control system gating triggers to volume or flow levels based on user input and provides a gating trigger signal to a magnetic resonance imaging (MRI) machine via a digital output channel from the A/D board, through a cable, to a gating input port of the MRI machine.

4. The system of claim 3, wherein said control system is configured to perform processing to at least one of minimize flow levels, adjust for a volume drift, reset a volume baseline, set pre trigger and post trigger delays for compensation to MR sequences, set monitoring alarms, or calculate spirometric parameters.

5. The system of claim 1, further comprising a mask and a pressure guide communicating said mask with said pneumotach.

6. The system of claim 1, said pneumotach further including a first pressure zone defined by said membrane and a first port, said first port including a first syringe tip.

7. The system of claim 6, said pneumotach further including a second pressure zone defined by said membrane and a second port, said second port including a second syringe tip.

8. The system of claim 1, said pneumotach further comprising a pressure sense chamber communicating with said airway coupler.

9. A system for monitoring a respiratory parameter including at least one of respiratory volume or respiratory flow, the system comprising:
a sensor for remotely detecting respiratory air having an air pressure, the sensor comprising:
a pressure chamber configured to remotely receive the respiratory air,
a membrane in the pressure chamber, the membrane movable responsive to the received air in the pressure chamber, and
an optical system configured to optically detect movement of the membrane and to optically transmit the detected movement; and
an electro-optic system for receiving the detected movement and converting the detected movement to an electrical movement measurement signal,
wherein the respiratory parameter is determined based on the movement measurement signal.

10. The system according to claim 9, further comprising:
a mask configured to receive the respiratory air from a patient; and
a pressure guide configured to transfer the respiratory air from the mask to the sensor.

11. The system according to claim 9, wherein the sensor includes a first port configured to communicate the respiratory air to the pressure chamber and a second port configured to communicate further air to the sensor.

12. The system according to claim 9, further comprising a control system for receiving the electrical movement measurement signal and determining the respiration parameter based on the electrical movement measurement signal.

13. The system according to claim 12, wherein the control system is configured to at least one of display waveforms associated with the determined respiration parameter, store the waveforms or calculate respirator parameters based on the electrical movement measurement signal.

14. The system according to claim 12, wherein the control system is configured to generate a gating trigger signal based on the determined respiration parameter and gating triggers set according to volume levels or flow levels, the control system providing the gating trigger signal to a magnetic resonance imaging (MRI) machine.

15. The system according to claim 9, wherein the optical system comprises at least one pair of optical fibers, one end of each of the at least one pair of fibers being arranged in proximity to the membrane, the at least one pair of optical fibers configured to transmit light from a light source to the membrane and detect light reflected from the membrane as the detected movement.

16. The system according to claim 15, wherein the electro-optic system includes the light source and a photodetector, the electro-optic system providing the light to the at least one pair of optical fibers and receiving the detected light from the at least one pair of optical fibers by the photodetector, the photodetector converting the detected light to the electrical movement measurement signal.

17. The system according to claim 15, wherein the pressure chamber includes first and second pressure chambers, the membrane includes first and second membranes in the respective first and second pressure chambers, and the at least one pair of fibers includes two pairs of fibers arranged in proximity to the respective first and second membranes.

18. The system according to claim 17, wherein the sensor includes a first port configured to communicate the respiratory air to the first pressure chamber, and a second port configured to communicate further air to the second pressure chamber.

19. A method for monitoring a respiratory parameter including at least one of respiratory volume or respiratory flow, the method comprising:
remotely receiving respiratory air having an air pressure in a pressure chamber including a membrane, the membrane moving responsive to the received air;
optically detecting movement of the membrane according to light reflected by the membrane;
optically transmitting the optically detected movement to a remote location;
receiving and converting the optically detected movement to an electrical movement measurement signal at the remote location; and
determining the respiratory parameter based on the electrical movement measurement signal.

20. The method according to claim 19, wherein the step of optically detecting the movement includes:
transmitting light from a light source to the membrane via a first optical fiber having an end arranged in proximity to the membrane; and
receiving the reflected light by a second optical fiber having an end arranged in proximity to the membrane.

21. The method according to claim 19, further including the steps of:
remotely receiving further air having a further air pressure in a further air pressure chamber including a further membrane, the further membrane moving responsive to the further air;
repeating the steps of optically detecting, optically transmitting, receiving and converting for movement of the further membrane in order to convert optically detected movement of the further membrane to a further electrical movement measurement signal; and
measuring a pressure difference between the air pressure and the further air pressure from the electrical movement measurement signal and the further electrical movement measurement signal.

22. The method according to claim 19, further including the step of:
remotely receiving further air having a further air pressure, the received further air being coupled to the received air in the pressure chamber in order to adjust for a pressure difference between the air pressure and the further air pressure.

23. The method according to claim 19, further including at least one of displaying waveforms associated with the determined respiration parameter, storing the waveforms or calculating respirator parameters based on the electrical movement measurement signal.

24. The method according to claim 19, further including:
setting gating triggers according to volume levels or flow levels;
generating a gating trigger signal based on the determined respiration parameter and the set gating triggers; and
providing the gating trigger signal to a magnetic resonance imaging (MRI) machine.

25. The method according to claim 19, further including processing the determined respiration parameter to at least one of minimize flow levels, adjust for a volume drift, reset a volume baseline, set pre trigger and post trigger delays for compensation to MR sequences, set monitoring alarms, or calculate spirometric parameters.
